(19) United States
(12) Patent Application Publication
(10) Pub. No.: US 2020/0297690 A1
(43) Pub. Date: Sep. 24, 2020

(54) SOLUBILIZED CANNABINOID POWDERS

(71) Applicant: Spyke, LLC, West Sacramento, CA (US)

(72) Inventor: Clinton Lee Crary, West Sacramento, CA (US)

(73) Assignee: Spyke, LLC, West Sacramento, CA (US)

(21) Appl. No.: 16/396,321

(22) Filed: Apr. 26, 2019

Related U.S. Application Data
(60) Provisional application No. 62/820,750, filed on Mar. 19, 2019.

(51) Int. Cl. A61K 31/352; A61K 9/16

(52) U.S. Cl. CPC A61K 31/352; A61K 9/1617; A61K 9/1652

(57) ABSTRACT

The invention provides anhydrous powderized water-soluble cannabinoid compositions, and methods for producing the same, which are redispersible upon re-hydration as a stable, homogenous emulsion for use in consumable food products.

### CROSS-REFERENCE TO RELATED APPLICATION

The present application claims the benefit of provisional application U.S. 62/820,750, filed Mar. 19, 2019, which is incorporated by reference in its entirety for all purposes.

### BACKGROUND

### Field of the Invention

The present invention relates generally to dry powders containing one or more water-soluble cannabinoid compounds. Also provided are products containing or produced from the dry powders, including aqueous beverage and other food compositions for human consumption. Methods for preparing the dry powders are provided.

### Description of Related Art

There are currently over 100 cannabinoid molecules identified within natural plants such as hemp and marijuana, collectively known as phytocannabinoids. Typically these cannabinoids are extracted using solvents from the biomass, then refined by distillation or chemical treatment to isolate the cannabinoid fraction. Additionally, the biosynthesis and/or synthesis of cannabinoids is also possible.

Cannabinoid concentrates, resins, and isolates are highly hydrophobic, and thus very insoluble in water. This property limits their absorption into tissues when consumed for medicinal or recreational purposes. Insolubility also limits their versatility and convenience as an ingredient in consumable products.

One approach to making cannabinoids water-soluble, and thus more highly bioavailable involves using physio-chemical solubilization techniques such as creating hydrocolloidal emulsions which form liposomes and micelle structures. These structures will encapsulate hydrophobic molecules, such as cannabinoids, thereby shielding them from the polar environment of water, and allow a stable, homogenous emulsion.

However, these structures can only exist while in a liquid, and are destroyed or fragment when dried out. The release of the captured cannabinoid lowers the stability of the cannabinoids if rehydrated, and effective the bioavailability of a food or beverage containing the broken structures. Therefore, it is key for an anhydrous formulation of cannabinoids to “re-form” its original liposomal or micellar structures as a redispersible emulsion, which will maintain its encapsulated, water-soluble properties upon re-hydration.

### CITATIONS TO BACKGROUND ART

U.S. Pat. No. 10,052,303
U.S. Pat. No. 10,046,018
U.S. Pat. No. 10,016,363
U.S. Pat. No. 9,861,611
U.S. Pat. No. 9,359,625
U.S. Pat. No. 9,095,555
U.S. Pat. No. 8,808,734
US Patent Appl. No. 2017/0816959 A1
US Patent Appl. No. 2017/0791181 A1
US Patent Appl. No. 2015/0165030 A1
Canadian Patent 2,507,688
Kiran, A. R. and Shyamsunder, R., Effect of Ultrasonication on Stability of Oil in Water Emulsion, International Journal of Drug Deliver, 3 (20111) 133-142.
Manecka, G. M., Formulation of Redispersible Freeze-dried Emulsions, EPSRC Centre for Innovative Manufacturing in Food, Annual Conference 23-24 Mar. 2016, Manufacturing Food Futures Holywell Park, Loughborough.
Freeze-Drying of Liposomes with Cryoprotectants and Its Effect on Retention Rate of Encapsulated Ftorafur and Vitamin A, Ze-Zhao Hua, Bao-Guo Li, Zhan-Jie Liu & Da-Wen Sun, Pages 1491-1505 I Published online: 6 Feb. 2007

### SUMMARY OF THE INVENTION

The invention provides anhydrous powderized water-soluble cannabinoid compositions, and methods for producing the same, which are redispersible upon re-hydration as a stable, homogenous emulsion for use in consumable food products. Within a water soluble, anhydrous powder, cannabinoids maintain a high-bioavailability as well as open up new delivery pathways for consumable products.

In one aspect, the invention provides an anhydrous water-soluble powder comprising a cannabinoid or mixture of cannabinoids present in the amount between 0.1% and 20%, optionally 1-10% or 1-5%, by weight of the dry powder.

The anhydrous powder may comprise any extract or resin of the cannabis plant, such as hemp or marijuana, that contains any amount of (a) natural cannabinoids, (b) biosynthetically-derived or manufactured cannabinoids, or (c) synthetically-manufactured cannabinoids.

In another aspect the powder includes a polyhydric alcohol, or mixture of polyhydric alcohols, such as erythritol, xylitol, or mannitol, present in the amount between 30% and 80%, optionally 40-60%, by weight of the dry powder.

In another aspect the powder includes a polysaccharide or other polymeric carbohydrate, or mixture of the same, such as maltodextrin present in the amount between 10% and 50%, optionally 20-40% by weight of the dry powder. The n polysaccharide can be linear, branched or cyclic. Such a polysaccharide can have e.g., 2 to 20 sugar units, optionally 3-20 or 3-17 sugar units. Some polysaccharides are a mixture of different chain lengths in which case the chain lengths can range from e.g., 2-20 units and/or have a mean chain length of 3-17, or 7-13 units.

In another aspect the powder includes a single class of phospholipids or a mixture of phospholipid surfactants, such as phosphatidylcholine or a mixture of amphiphilic lecithin extracts, present in the amount between 0.1% and 10%, optionally 1-5%, by weight of the dry powder.

In another aspect the powder includes an amino acid, or mixture of multiple amino acids, such as glycine, alanine, leucine, or other amino acid with a hydrophobic side chain, in the amount between 0.1% and 10%, optionally 1-5% by weight of the dry powder.

Anhydrous powders can include any or all of the above aspects as well as a cannabinoid.

Additional ingredients may improve the solubility and utility of the anhydrous powder, such as a mixture of medium-chain triglycerides (containing over 50 or 75% chain lengths of C6, C8, C10, and/or C12), present in the amount between 0.1% and 5%, optionally 1-3% by weight of the dry powder, a monoglyceride, present in the amount between 0.1% and 5%, optionally 1-3%, by weight of the dry powder, triglyceride or mixture of triglycerides, wherein the mixture of triglycerides is medium-chained triglycerides (MCT) derived from coconut oil, and is present at 0.1% to 5%, optionally 1-3% by dry weight of the anhydrous powder, a natural gum present in the amount between 0.1% to 3%, optionally 0.5-2% by dry weight of the anhydrous powder; a bicarbonate, present at 0.1% to 5%, optionally 1-3%, by dry weight of the anhydrous powder; a natural mineral powder, present in the amount between 0.1% and 3%, optionally 1-3%, by weight of the dry powder. A medium chain triglyceride means a mean chain length of fatty acids from 6 to 12 carbon atoms.

The invention also provides water-soluble powder containing one or more cannabinoids that is anhydrous after a drying process to include (a) sublimation (also known as freeze-drying or lyophilization), (b) spray drying, as described in Perry's Chemical Engineering Handbook, pp. 20-57, 6^{th} ed., 1984, or (c) supercritical drying, including but not limited to carbon dioxide or Freon as solvents.

The invention provides a water-soluble anhydrous powder comprising a cannabinoid or mixture of cannabinoids, a polyhydric alcohol, a polysaccharide, a phospholipid, and an amino acid. Optionally, the cannabinoid comprises any of tetrahydrocannabinol (THC), cannabidiol (CBD), cannabigerol (CBG), cannabinol (CBN), or other plant-derived cannabinoid, or any combination thereof. Optionally, the cannabinoid comprises any of tetrahydrocannabinol (THC), cannabidiol (CBD), cannabigerol (CBG), cannabinol (CBN), or other biosynthetically-derived cannabinoid, or any combination thereof. Optionally, the cannabinoid comprises any of tetrahydrocannabinol (THC), cannabidiol (CBD), cannabigerol (CBG), cannabinol (CBN), or other synthetically-derived cannabinoid, or any combination thereof. Optionally, the polyhydric alcohol is erythritol, xylitol, or mannitol, the polysaccharide is maltodextrin, the phospholipid is phosphatidylcholine or a mixture of amphiphilic lecithin extracts, and the amino acid is glycine, alanine, leucine, or other amino acid with a hydrophobic side chain. Optionally, the cannabinoid or mixture of cannabinoids is present at 0.1% to 20% by dry weight of the anhydrous powder, the polyhydric alcohol is present in the amount between 30% and 80%, between 35% and 75%, between 40% and 70%, optionally 40-60%, by weight of the anhydrous powder, the polysaccharide is present in an amount between 10% and 50%, between 15% and 45%, between 20% and 40%, optionally 20-40%, by weight of the anhydrous powder, the phospholipid is present in the amount between 0.1% and 10%, between 0.5% and 8%, between 1% and 6%, optionally 2-5% by weight of the anhydrous powder, and the amino acid is present in the amount between 0.1% and 10%, between 0.5% and 8%, between 1% and 6%, optionally 1-4%, by weight of the anhydrous powder. Optionally, the powder further comprises a monoglyceride, wherein the monoglyceride is glycerol monostearate, and is present at 0.1% to 5% by dry weight of the anhydrous powder. Optionally, the powder further comprises a triglyceride or mixture of triglycerides, wherein the mixture of triglycerides is medium-chain triglycerides (MCT) derived from coconut oil, and is present at 0.1% to 5% by dry weight of the anhydrous powder. Optionally, the powder, further comprising a natural gum, wherein the gum is acacia gum and is present at 0.1% to 3% by dry weight of the anhydrous powder. Optionally, the powder further comprises a bicarbonate, wherein the bicarbonate is sodium bicarbonate and is present at 0.1% to 5% by dry weight of the anhydrous powder. Optionally, the powder further comprises a powdered mineral, wherein the powdered mineral is powdered silica and is present at 0.1% to 3% by dry weight of the anhydrous powder. Optionally, the powder further comprises one or more flavoring agents, colored dyes, sweeteners, or stabilizing agents. Optionally, the powder is combined with water or other consumable liquid. Optionally, the powder is combined with food or other consumable food ingredient. Optionally, the powder has a solubility greater than 25% by weight of the dry powder in water with the cannabinoid mixture present at greater than 5% by dry weight of the anhydrous powder. Optionally, the powder has a solubility greater than 30% by weight of the dry powder in water, with the cannabinoid mixture present at greater than 4% by dry weight of the anhydrous powder. Optionally, the powder has a solubility greater than 35% by weight of the dry powder in water, with the cannabinoid mixture present at greater than 3% by dry weight of the anhydrous powder. Optionally, the powder is prepared by solubilizing an extract comprising a cannabinoid or mixture of cannabinoids, in a mixture combining the solubilized cannabinoids with water, a polyhydric alcohol, a polysaccharide, a phospholipid, and an amino acid, and drying. Optionally, the drying is performed by lyophilization.

The invention further provides a method of preparing a powder as described above, comprising solubilizing an extract comprising a cannabinoid or mixture of cannabinoids, in a mixture combining the solubilized cannabinoids with water, a polyhydric alcohol, a polysaccharide, a phospholipid, and an amino acid, and drying. Optionally the method further comprises rehydrating the anhydrous powder. Optionally, the method further comprises combining the anhydrous powder of a hydrated form thereof with water or other consumable liquid. Optionally the method further comprises combining the anhydrous powder of a hydrated form thereof with food or other consumable food ingredient.

These and other features and advantages of this invention are described in, or are apparent from, the following detailed description of various exemplary embodiments of the apparatus and methods according to this invention.

### DETAILED DESCRIPTION OF THE INVENTION

Unless defined otherwise, all technical and scientific terms used herein have the same meaning as is commonly understood by one of skill in the art to which the invention(s) belong. All patents, patent applications, published applications and publications, Genbank sequences, databases, websites and other published materials referred to throughout the entire disclosure herein, unless noted otherwise, are incorporated by reference in their entirety for all purposes. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information. Unless otherwise apparent from the context any aspect, embodiment, feature, element, or step can be used in combination with any other. Disclosure of a range should be understood as also all integers and subranges of integers within the range. Reference to a composition comprising one or more stated ingredients should additionally be taken as a disclosure of a composition consisting of or consistent essentially of the stated ingredients.

As used herein, “cannabinoid” refers a class of diverse chemical compounds that act on cannabinoid receptors in cells that alter neurotransmitter release in the brain. Ligands for these receptor proteins include the endocannabinoids (produced naturally in the body by animals) the phytocannabinoids (found in some plants), and synthetic cannabinoids (manufactured artificially). The most widely known cannabinoids found in cannabis known to have the most therapeutic properties are tetrahydrocannabinol (THC) and cannabidiol (CBD). A number of other cannabinoids, such as cannabigerol (CBG) and cannabinol (CBN), also have been shown to exhibit health benefits. There are at least 113 different cannabinoids isolated from cannabis, exhibiting varied effects.

The, the term “cannabinoid” may refer to either a natural or synthetic cannabinoid. Synthetic cannabinoids encompass a variety of distinct chemical classes: the classical cannabinoids structurally related to THC, the non-classical cannabinoids (cannabimimetics) including the aminoalkylindoles, 1,5-diarylpyrazoles, quinolones, and aryl sulfonamides as well as eicosanoids related to endocannabinoids. Processes for the synthesis of various cannabinoid compounds are described in U.S. Pat. No. 9,359,625 and others.

As used herein, cannabidiol (CBD) refers to a non-intoxicating cannabinoid found in cannabis. After tetrahydrocannabinol (THC), cannabidiol (CBD) is the second-most abundant cannabinoid in the plant, and has many potential therapeutic benefits, including anti-inflammatory, analgesic, anti-anxiety and seizure-suppressant properties. Cannabidiol can be sourced from both marijuana plants and hemp plants.

As used herein, micellar or liposomal solubilization (or solubilization) is the process of incorporating the solubilizate (the component that undergoes solubilization) into micelles or liposomes. Solubilization may occur in a system consisting of a solvent, an association colloid (a colloid that forms micelles or liposomes), and at least one other solubilizate. Solubilization is distinct from dissolution because the resulting fluid is a colloidal dispersion involving an association colloid. This suspension is distinct from a true solution, and the amount of the solubilizate in the micellar or liposomal system can be different (often higher) than the regular solubility of the solubilizate in the solvent.

In non-chemical literature and in everyday language, the term “solubilization” is also sometimes used in a broader meaning as “to bring to a solution or (non-sedimenting) suspension” by any means, e.g., leaching by a reaction with an acid.

As used herein, a “water-soluble anhydrous powder”, refers to the compositions that contain mixtures of water-soluble forms cannabinoids after the drying process that has removed most or all water.

The term “sonication” is the act of applying sound energy to agitate particles in a sample, for various purposes such as the extraction of multiple compounds from plants, microalgae and seaweeds. Ultrasonic frequencies (>20 kHz) are usually used, leading to the process also being known as ultrasonication.

The term “lecithin” is used in the generic sense to designate any group of yellow-brownish fatty substances occurring in animal and plant tissues, which are amphiphilic they attract both water and fatty substances (and so are both hydrophilic and lipophilic), and are used for smoothing food textures, dissolving powders (emulsifying), homogenizing liquid mixtures, and repelling sticking materials. More particularly, lecithins are mixtures of glycerophospholipids including phosphatidylcholine, phosphatidylethanolamine, phosphatidylinositol, phosphatidylserine, and phosphatidic acid.

A major source of lecithin is soybean oil. Because of the EU requirement to declare additions of allergens in foods, in addition to regulations regarding genetically modified crops, a gradual shift to other sources of lecithin (e.g., sunflower oil) is taking place. The main phospholipids in lecithin from soya and sunflower are phosphatidyl choline, phosphatidyl inositol, phosphatidyl ethanolamine, phosphatidylserine, and phosphatidic acid. They often are abbreviated to PC, PI, PE, PS and PA, respectively.

The term “lecithin extract” is used to describe one or more phospholipids purified from lecithin.

As used herein the terms “freeze-drying” and “lyophilization” are considered synonymous, and mean any of a number of known water removal processes applied to materials. In particular, many such processes are known for the preservation of perishable materials, e.g., to extend shelf life or make the material more convenient for transport. Freeze-drying works by freezing the material, then reducing the pressure to allow the frozen water in the material to sublimate in a process of sublimation. The process of sublimation is often aided by adding heat.

Solubility refers to the number of grams of dried powder as described herein dissolvable in 100 g water measured at 25° C. Optionally, the water-soluble anhydrous powder has a solubility greater than 25% (and optionally up to, e.g., 30, 40, 50, 75 or 100%) by weight of the dry powder in water with the cannabinoid mixture present at greater than 5% by dry weight of the anhydrous powder. Optionally, the powder having a solubility greater than 30% (and optionally up to, e.g., 40, 50, 75 or 100%) by weight of the dry powder in water, with the cannabinoid mixture present at greater than 4% by dry weight of the anhydrous powder.

According to the present invention, cannabinoids are subject to a process whereby they are first solubilized using an amphiphilic phospholipids or a mixture of phospholipid compounds, such as phosphatidylcholine, and water, and then combined, with a polyhydric alcohol, such as erythritol, a powdered long-chain polysaccharide such as maltodextrin, and an amino-acid to stabilize the hydrophobic compounds within the liposomal structures, such as glycine. The wet liquid is then subjected to lyophilization to very low moisture content, preferably less than 3% water and most preferably less than 1% water, measured by the weight of the dried product, with the resulting dried cake of material pulverized into the final anhydrous powder.

The water-soluble cannabinoid-rich liquid is then ready to be mixed with the any or all of the following required or optional ingredients including, but not necessarily restricted to:

(a) a mixture of polyhydric alcohols, preferably erythritol, xylitol, or mannitol, present in the amount between 30% and 80%, between 35% and 75%, between 40% and 70%, optionally 40-60%, by weight of the anhydrous powder;
(b) a long-chain polysaccharide or other polymeric carbohydrate, or mixture of the same, preferably Tapioca Maltodextrin, present in the amount between 10% and 50%, between 15% and 45%, between 20% and 40%, optionally 20-40%, by weight of the anhydrous powder;
(c) a phospholipids or a mixture of phospholipid surfactants, preferably phosphatidylcholine, present in the amount between 0.1% and 10%, between 0.5% and 8%, between 1% and 6%, optionally 2-5% by weight of the anhydrous powder;
(d) an amino acid, or mixture of multiple amino acids, preferably glycine, present in the amount between 0.1% and 10%, between 0.5% and 8%, between 1% and 6%, optionally 1-4%, by weight of the anhydrous powder. The anhydrous powder can be mixed with one or more flavoring agents, colored dyes, sweeteners, stabilizing agents, scents, bulking agents, cryoprotectants and the like. The dried powder with or without such additional agents can then be added to a food or drink or combined with one or more other food ingredients to form a food or drink.

### Example 1—THC1

| Mixture A (Powders): | Weight | | % Wet | % Dry |
|---|---|---|---|---|
| Polyhydric alcohol | 50.0 | grams | 43.5% | 74% |
| [Erythritol] | | | | |
| Polysaccharide [Maltodextrin] | 7.5 | grams | 6.5% | 10.5% |
| Amino acid [Glycine] | 1.0 | grams | 1.0% | 1.5% |
| Total | 58.5 | grams | 51% | 86% |

Mixture A is prepared by blending to a homogenous powder.

The percent dry is the final anhydrous powder after drying.

| Mixture B (liquids): | | | | |
|---|---|---|---|---|
| THC distillate/extract | 2.8 | grams | 2.5% | 4.1% |
| (~71% THC) | | | | |
| Phospholipid | 5.0 | grams | 4.5% | 6.6% |
| [Phosphatidylcholine] | | | | |
| Water (heated) | 48.7 | grams | 42% | <1% |
| Total | 56.5 | grams | 49% | ~11% |
| Grand Total (wet) | 115.0 | grams | | |
| Grand Total (dried) | 67.7 | grams | | |

Mixture B was sonicated at 1000 W for 3 minutes.

Mixture B was mixed into Mixture A using a high-sheer mixer, until it was a homogenous slightly yellow white liquid.

The resulting wet mixture was placed onto a metal sheet tray, with 113.5 g mixture onto tray after mechanical loss.

The combined mixture was then freeze dried for a 24-hour cycle, with 67.7 grams recovered as a dry powder, named Sample 1. Therefore, ˜59.6% of the starting mixture prior to freeze-drying, was recovered, with a 40.4% loss of water.

There was 45.8 grams of loss from water out of an original 47.7 grams of water, reflecting a loss of 96% of the water added.

The THC content was expected to be approximately 2.94%, based on 71% of 2.8 grams THC distillate, divided by the 67.7 g total weight recovered after drying. Sample 1 was analyzed by Steep Hill Labs in Berkeley, Calif. on Mar. 8, 2019, and found to be 3.39% THC. The difference of 0.45% represented the lab tested Sample 1 at 15% higher than expected, but may be due to instrument calibration.

### Example 1 Solubilization Test

Sample 1 was subjected to a solubilization test to see how efficiently the sample powders solubilized in water. Small (0.25 g) amounts of Sample 1 were added to 25 degree Celsius water, with mixing after each addition by 3-seconds of shaking the vial by hand. The addition of more of Sample 1 continued until the first sign that powder began to drop out of the solution, thereby reaching the maximum solubility. After repeated solubility tests, Sample 1 averaged a solubility of 4.15 g in the 8.0 grams of water, yielding a 34% solubility in 25 C water. [4.15 g/12.15 g=0.342] The cannabis extract typically contains approximately 20% to 30% non-polar, non-THC plant compounds, such as resins and polyphenols, all of which have extremely low solubility in water. The proportions of these impurities will have a slight affect the solubilization of the final product.

Using the amino acid to protect the cannabinoid-containing liposomal or micellar structures from damage due to ice crystals or other trauma during dehydration, and aiding the re-formation of the structures during the re-hydration process is a critical new ingredient in the overall process that allows the cannabinoids to remain solubilized. This ability provides the basis for the inclusion of non-polar cannabinoids in powdered drink mixes and other consumable products. The invention thus provides a powder containing water-soluble cannabinoids, created from a mixture of one or more of sugar-alcohols, polysaccharides, lecithin extracts as surfactants, an amino acid, water are dehydrated and form a water-soluble dry powder intended as a finished product or ingredient in various consumable products.

Methods for preparing the water-soluble powder include mixing of liquid and dry ingredient compositions together in any order prior to the drying process are also provided. Methods for preparing the water-soluble powder include the initial preparation of the water-soluble cannabinoid concentrate through the mixing of the cannabinoid, as an insoluble distillate or a concentrated distillate fraction, with the surfactant(s) and water, thus creating a water-soluble cannabinoid-rich liquid. This water-soluble liquid concentrate would optionally have undergone a treatment of energy such as ultra-sonication, in order to increase the formation of liposomal or micellar structures which encapsulate the insoluble cannabinoids while in a liquid suspension.

The water-soluble cannabinoid-rich liquid is then ready to be mixed with the required or optional ingredients including, but not necessarily restricted to: a polyhydric alcohol, present in the amount between 30% and 80% by weight of the dry powder; a long-chain polysaccharide or other polymeric carbohydrate, or mixture of the same, present in the amount between 0.1% and 30% by weight of the dry powder; a single class of phospholipids or a mixture of phospholipid surfactants, present in the amount between 0.1% and 10% by weight of the dry powder; and an amino acid, or mixture of multiple amino acids, present in the amount between 0.1% and 10% by weight of the dry powder.

For the purpose of improving solubility of the cannabinoid mixture, improving taste, or decreasing the likelihood of clumping or caking, additional ingredients may comprise the water-soluble anhydrous powder, including a triglyceride mixture, present in the amount between 0.1% and 5% by weight of the dry powder; a monoglyceride, present in the amount between 0.1% and 5% by weight of the dry powder; a natural gum present in the amount between 0.1% to 3% by dry weight of the anhydrous powder; a bicarbonate, present at 0.1% to 5% by dry weight of the anhydrous powder; a natural mineral powder, present in the amount between 0.1% and 3% by weight of the dry powder.

1. A water-soluble anhydrous powder comprising a tetrahydrocannabinol, a polyhydric alcohol, a polysaccharide, a phospholipid, and an amino acid, wherein the polyhydric alcohol is erythritol, xylitol, or mannitol, the polysaccharide is maltodextrin, the phospholipid is phosphatidylcholine or a mixture of amphiphilic lecithin extracts, and the amino acid is glycine, alanine, leucine, or other amino acid with a hydrophobic side chain, wherein the cannabinoid or mixture of cannabinoids is present at 0.1% to 20% by dry weight of the anhydrous powder, the polyhydric alcohol is present in the amount between 30% and 80%, by weight of the anhydrous powder, the polysaccharide is present in an amount between 10% and 50%, between 15% and 45%, the phospholipid is present in the amount between 0.1% and 10%, and the amino acid is present in the amount between 0.1% and 10%, by weight of the anhydrous powder.

2-4. (canceled)

5. The water-soluble anhydrous powder in claim 1, wherein the polyhydric alcohol is erythritol, xylitol, or mannitol, the polysaccharide is maltodextrin, the phospholipid is phosphatidylcholine or a mixture of amphiphilic lecithin extracts, and the amino acid is glycine, alanine, leucine, or other amino acid with a hydrophobic side chain.

6. The water-soluble anhydrous powder in claim 1, wherein the polyhydric alcohol is present in an amount between 40-60%, by weight of the anhydrous powder, the polysaccharide is present in an amount between 20-40%, by weight of the anhydrous powder, the phospholipid is present in an amount between 2-5% by weight of the anhydrous powder, and the amino acid is present in an amount between 1-4%, by weight of the anhydrous powder.

7. The water-soluble anhydrous powder in claim 1, further comprising a monoglyceride, wherein the monoglyceride is glycerol monostearate, and is present at 0.1% to 5% by dry weight of the anhydrous powder.

8. The water-soluble anhydrous powder in claim 1, further comprising a triglyceride or mixture of triglycerides, wherein the mixture of triglycerides is medium-chain triglycerides (MCT) derived from coconut oil, and is present at 0.1% to 5% by dry weight of the anhydrous powder.

9. The water-soluble anhydrous powder in claim 1, further comprising a natural gum, wherein the gum is acacia gum and is present at 0.1% to 3% by dry weight of the anhydrous powder.

10. The water-soluble anhydrous powder in claim 1, further comprising a bicarbonate, wherein the bicarbonate is sodium bicarbonate and is present at 0.1% to 5% by dry weight of the anhydrous powder.

11. The water-soluble anhydrous powder in claim 1, further comprising a powdered mineral, wherein the powdered mineral is powdered silica and is present at 0.1% to 3% by dry weight of the anhydrous powder.

12. The water-soluble anhydrous powder in claim 1, further comprising one or more flavoring agents, colored dyes, sweeteners, or stabilizing agents.

13. The water-soluble anhydrous powder in claim 1, which is combined with water or other consumable liquid.

14. The water-soluble anhydrous powder in claim 1, which is combined with food or other consumable food ingredient.

15. The water-soluble anhydrous powder in claim 1, having a solubility greater than 25% by weight of the dry powder in water with the cannabinoid mixture present at greater than 5% by dry weight of the anhydrous powder.

16. The water-soluble anhydrous powder in claim 1, having a solubility greater than 30% by weight of the dry powder in water, with the cannabinoid mixture present at greater than 4% by dry weight of the anhydrous powder

17. The water-soluble anhydrous powder in claim 1, having a solubility greater than 35% by weight of the dry powder in water, with the cannabinoid mixture present at greater than 3% by dry weight of the anhydrous powder.

18. The water-soluble anhydrous powder in claim 1, prepared by solubilizing an extract comprising a cannabinoid or mixture of cannabinoids, in a mixture combining the solubilized cannabinoids with water, a polyhydric alcohol, a polysaccharide, a phospholipid, and an amino acid, and drying.

19. The water-soluble anhydrous powder in claim 1, wherein the drying is performed by lyophilization.

20. A method of preparing the powder of claim 1, comprising solubilizing an extract comprising a cannabinoid or mixture of cannabinoids, in a mixture combining the solubilized cannabinoids with water, a polyhydric alcohol, a polysaccharide, a phospholipid, and an amino acid, and drying.

21. A method of claim 20, further comprising rehydrating the anhydrous powder.

22. A method of claim 20, further comprising combining the anhydrous powder of a hydrated form thereof with water or other consumable liquid.

23. A method of claim 20, further comprising combining the anhydrous powder of a hydrated form thereof with food or other consumable food ingredient.
